(19) United States
(12) Patent Application Publication
(10) Pub. No.: US 2005/0059920 A1
(43) Pub. Date: Mar. 17, 2005

(54) Medical foot helper for diabetic, arthritic, disabled, elderly and obese persons

(76) Inventor: Rebecca Anne Baril, Chicago, IL (US)

Correspondence Address: DR. REBECCA A. BARIL, D.P.M., # 701-B, 1455 N. CLARK STREET, CHICAGO, IL, 60610, US

(21) Appl. No.: 10/661,417

(22) Filed: Sep. 15, 2003

(51) Int. Cl. A61M 35/00

(52) U.S. Cl. 604/1

(57) ABSTRACT

An essential health care instrument for the ankle, foot and toes which assists in the foot care of diabetic, arthritic, disabled, elderly and obese persons. The length of this lightweight instrument enables visual inspection of areas subject to bacteria and infection without assistance and eliminates reaching or bending. The rod enables user(s) to medicate tissues surrounding their feet and digits (toes) from a standing, sitting or lying position. One end of this instrument provides a way for self inspection for ulcers, sores, cuts or sanitary problems of the feet. This process is performed by use of the magnifying mirror soldered to the end of the instrument rod. Another end of this lightweight instrument enables a sanitation process and medicine application for foot surfaces and between the toes. The cleansing and application of medicine is achieved through the use of washable and reusable sponges attached to the end of the rod.

### BACKGROUND OF THE INVENTION

My invention of “The Medical Foot Helper for Diabetic, Arthritic, Disabled, Elderly and Obese Persons” is a welcomed and desperately needed instrument. My instrument will provide the previously mentioned individuals with the ability to solve many of their visual and medical problems related to their feet, ankles, and digits (toes) without extensive reaching and bending.

As a Doctor of Podiatric Medicine, in the hospital and in private practice, I have encountered persons without the ability or the flexibility to self-examine, self-sanitize or self-medicate their own feet, ankles or digits (toes). These persons have varying degrees of medical needs for the surfaces of their feet, ankles and between their digits (toes). Their daily needs include inspection and care of bacteria, infections, ulcers, cuts, sores, tissue debris and/or proper sanitation needs. Due to various medical and physical conditions and/or limitations, many of these same individuals are physically unable to reach or bend either their backs, their arms, at the waist, or at the knees, therefore, making inspection or impossible or inadequate. “The Medical Foot Helper for Diabetic, Arthritic, Disabled, Elderly and Obese Persons” will assist these individuals with an instrument that has the ability to extend their reach, approximately three (3) feet, while one end is providing a magnified mirrored glass that will allow up close visual examination from a distance, while the other end is providing an applicator tip with washable, reusable applicator sponges to absorb liquids, remove tissue debris, sanitize from a distance and the sponges will also absorb medications & creams for applications from a distance.

Most diabetic individuals, for example, are susceptible to ulcers, bacteria and infections of the feet and digits (toes). On a daily basis, diabetic individuals must examine the top and sides of their feet, between each digit (toe) and the bottom surface of each foot. Due to illnesses, arthritis, various joint diseases and obesity for example, a large percentage of these persons can not reach the tops, sides or bottoms of their feet. Due to many physical and medical challenges, many individuals can not get out of bed or rise from a lying position. In addition, many individuals can not rise from a sitting position with or without assistance. Therefore, these same individual can not reach or bend to visually inspect or medically treat their feet. They have no ability to perform even the basic self-examination or self-treatment of their own feet, ankles and digits (toes).

I have witnessed patients attempting to use hand held swabs, pads, pencils and wire hangers to perform their health care needs. Most individuals were not flexible enough to even reach their feet with these short objects. If the reach was made, usually the swabs, pads or pencils would fall, and since they are not sterile, then when used regardless they caused dirt and germs to enter the ulcers or open wound. If the reach was successful, the process was not successful as the swabs can not contain enough medication for proper coverage. In addition, swabs and pads did not have the absorbency to remove liquid from infection or tissue debris. Persons using pencils and reaching the feet would tear the tissue, sustain cuts and have lead particles break off into the ulcer or open wound. The use of hangers would also cause tears in the tissues, cuts, and infections. Currently there is no instrument to safely inspect and treat medical conditions related to the feet, ankle and digits (toes) without assistance and without excessive reaching and/or bending. Therefore, I feel “The Medical Foot Helper for Diabetic, Arthritic, Disabled, Elderly and Obese Persons” will solve the problem of needing to reach the feet to apply medicine, since it has the extended rod with the sponge applicator tip at one end. Also, it will solve the problem of being unable to do daily inspections of the feet due to inabilities in bending, since it has a magnified mirror at the other end of the extended rod to visualize all sides of the feet.

### BRIEF SUMMARY OF THE INVENTION

My invention, “The Medical Foot Helper for Diabetic, Arthritic, Disabled, Elderly and Obese Persons”, is an instrument which enables the self-examination, self-sanitation, self-medication of the feet, ankle, digits (toes) and surrounding tissues. Diabetic, arthritic, disabled, elderly and obese persons, for example, may have physical and medical conditions which make their ability to bend and reach extremely difficult, if not impossible. This prevents many of these persons from visually examining, sanitizing, or medicating their feet, ankles and digits (toes). The length of this lightweight instrument enables visual inspection of areas subject to bacteria and infection without assistance and eliminates reaching or bending. The rod enables user(s) to medicate tissues surrounding their feet and digits (toes) from a standing, sitting or lying position.

One end of this instrument provides a method to self-examination for ulcers, sores, cuts or sanitary problems of the feet. This process is performed by use of the magnifying mirror soldered to the end of the instrument rod. The other end of this lightweight instrument enables a sanitation process and medicine application for foot surfaces and between the toes. The cleansing and application of medicine is achieved through the use of washable and reusable sponges attached to the end of the rod.

Patients, with bending, reaching, physical and/or medical restrictions, have use hand held swabs, pads, pencils and wire hangers to perform their foot health care needs. Since the swabs, pads or pencils are difficult to grip, many times they have fallen, and not being sterile, have caused dirt and germs to enter ulcers or open wounds. Swabs and pads can not absorb all medicines and creams for proper coverage. Swabs and pads do not have the absorbency to remove excess liquid from infection or tissue debris. The use of wire or plastic hangers, pencils will cause tears in the tissues, produce cuts, and cause infections.

There is no instrument to safely inspect and treat medical conditions related to the feet, ankle and digits (toes) without assistance and without excessive reaching and/or bending. “The Medical Foot Helper For Diabetic, Arthritic, Disabled, Elderly and Obese Persons”, having an extended length rod with attached sponge applicator at one end, will enable the sterilization, sanitation, and the self application of medicines to the feet, surrounding tissues, ankles and between digits (toes). My instrument will solve the difficulties and inabilities, for persons with physical and medical restrictions, to perform daily inspections of the feet, surrounding tissues, ankles and digits (toes) due to required need to bend and reach. My instrument, having an extended length rod with attached magnified mirror at the other end, will enable the user(s) to visually examine foot tissues, between each digit (toe) and the tops, bottoms, sides of their feet from a distance, without bending and reaching.

### BRIEF DESCRIPTION OF THE SEVERAL VIEWS OF THE DRAWING

FIG. 1—This drawing depicts the overall view of “The Medical Foot Helper for Diabetic, Arthritic, Disabled, Elderly and Obese Persons”, in its entirety. In this illustration, my invention is viewed from the side, from top to bottom, which is approximately three (3) feet in length in its entirety. All parts of this instrument are depicted this includes one (1) instrument rod, one (1) rubber grip, one (1) magnified mirror glass, one (1) frame for mirror placement, one (1) circular hinge for frame attachment to the rod, one (1) hinge for applicator tip attachment to the rod, one (1) applicator tip, one (1) small size applicator sponge, and two (2) circular discs.

FIG. 2—This drawing depicts an aerial view of one (1) magnified glass mirror, one (1) frame to secure mirror, one (1) hinge for mirror to attach to instrument rod, a portion of one (1) instrument rod, a portion of one (1) rubber grip. In this illustration, one end of my invention is viewed from a close distance.

FIG. 3—This drawing depicts a side view one (1) applicator tip, one (1) small size applicator sponge, two (2) circular discs to secure applicator sponge, one (1) hinge to attach applicator tip to instrument rod, a portion of one (1) instrument rod, a portion of one (1) rubber grip. In this illustration, one end of my invention is viewed from a close distance.

FIG. 4—This drawing depicts a side view one (1) applicator tip, one (1) small size applicator sponge, two (2) circular discs to secure applicator sponge, one (1) hinge to attach applicator tip to instrument rod, and a portion of one (1) instrument rod. In this illustration, one end of my invention is viewed from a close distance.

FIG. 5—This drawing depicts a side view of one (1) large sized applicator sponge. In this illustration, one (1) large size applicator sponge is viewed from a close distance.

FIG. 6—This drawing depicts a side view of one (1) individual using “The Medical Foot Helper For Diabetic, Arthritic, Disabled, Elderly and Obese Persons”, in its entirety, while sitting in a chair with her foot lowered. This view illustrates my invention being used at a near vertical angle. In this view, the subject is utilizing the magnified glass mirror for visual inspection of the foot from a distance.

FIG. 7—This drawing depicts a side view of one (1) individual using “The Medical Foot Helper For Diabetic, Arthritic, Disabled, Elderly and Obese Persons”, in its entirety, while lying in bed. This view illustrates my invention being used at a near horizontal angle. In this view, the subject is utilizing the applicator sponge and applicator tip.

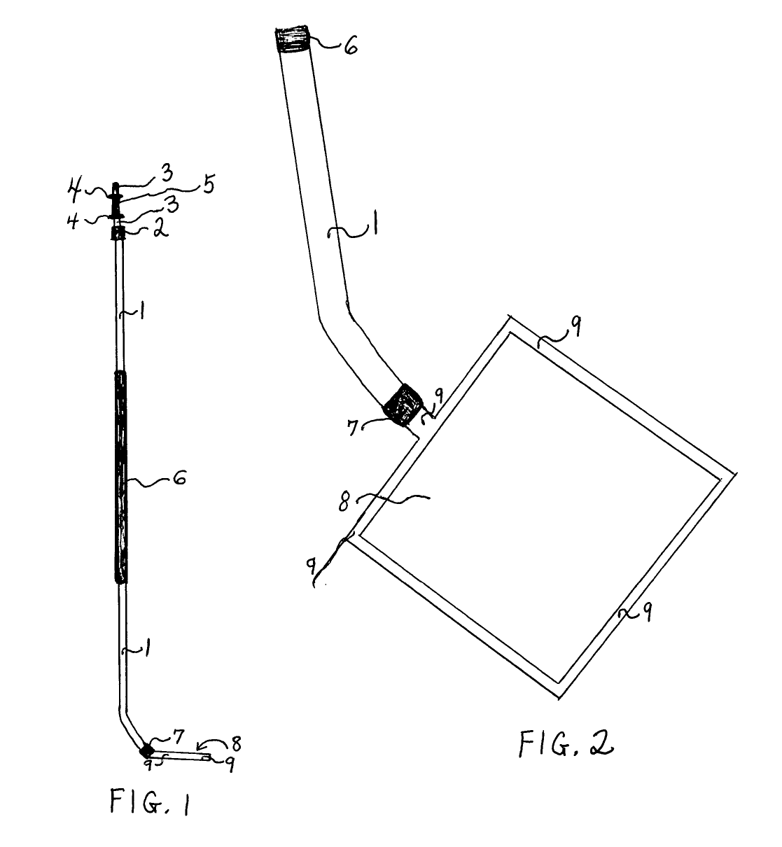

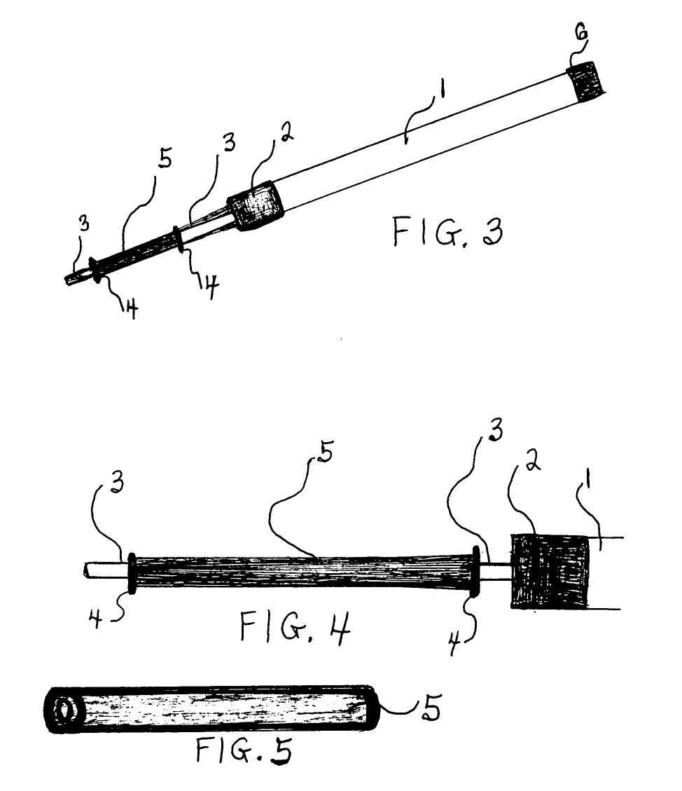

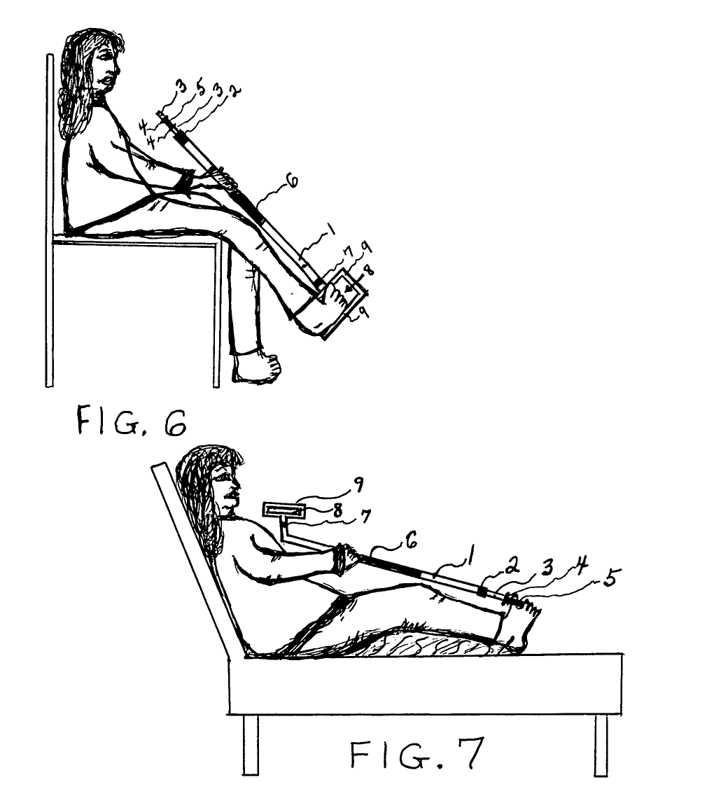

### DETAILED DESCRIPTION OF THE INVENTION

The base rod of the instrument has the length to enable the examination and/or the user(s) to self-examine the tops, sides and bottoms of his/her feet and in between each digit (toe) from an upright standing position, a sitting position and/or in a lying position. The base rod of the instrument is composed of a lightweight titanium type metal and/or a lightweight composite material which would provide the strength to enable the user(s) to manually press the instrument against his/her foot surfaces, on & in between the digits (toes), and on & around the ankle, with such pressure so as to not have the instrument rod bend. The rod has the strength to withstand the manual pressure applied by the user(s) to enable the user(s) to perform the manual cleaning of debris tissues and/or sores on the foot or in between the toes, manual dermabrasion and manual application of medicines surrounding the feet and/or in between the digits (toes). This same instrument rod has a magnifying mirror permanently attached to allow the user(s) the ability to self-inspect the top, sides & bottoms of the feet (all surfaces). In addition, this attached magnifying mirror allows the user(s) to visually self-examine his/her digit (toes), both in between each digit (toe) and/or on the bottom(s).

The soft rubber type grip material has been placed in the center area of the instrument rod to allow the user(s) to position his/her hands and fingers on top of this material regardless of which end of the instrument is being used by said user(s). This soft grip material will enable the user(s) to grasp and maneuver this instrument rod without the rod sliding, therefore, losing his/her grip and/or maneuver ability. This material will allow the user(s) with arthritis or other diseases of the fingers, hands and/or joints to more comfortably grasp and maneuver this instrument.

The magnifying mirrored glass is placed within an attached frame and is soldered to a hinge which, in turn, has been soldered to one end of the instrument rod. The mirror is composed of magnified glass in such a size, approximately 6″×8″, as to allow the user(s) to obtain visual inspection from a distance either in a standing, sitting and/or lying position. This magnifying mirror provides the remedy to inspect and detect ulcers, sores, cuts, abrasions, tissue debris and/or sanitation problems between and around the digits (toes) from a distant standing, sitting and/or lying position. In addition, this mirror will enable the user(s) to obtain a complete magnified visual self-examination of the top, sides and bottoms of each foot.

The applicator tip is composed from a lightweight metal and/or composite and is soldered to a hinge which, in turn, has been soldered to one end of the instrument rod. The purpose of the applicator tip is to hold in place the applicator sponge and to bear the manual pressure while the applicator sponge is in use. The applicator tip is small enough in diameter to enable the user(s) to maneuver both the tip and sponge underneath and between each digit (toe).

The detachable sponge applicator is composed of a lightweight sponge type material with a durability factor to allow for its washing and reuse. The applicator sponge is placed over the applicator tip which has been soldered to one end of the instrument rod. The detachable sponge applicator is composed of a lightweight material that will allow for the absorption of medicines and creams. This same sponge applicator has the strength and durability to allow for the dermabrasion of bacteria and/or debris tissues of the user(s). The sponge will be manufactured in two (2) diameters/widths: The first diameter having a larger width for area use on the user(s) ankle and/or foot surface. The second diameter, having a remarkably smaller width, enables the user(s) to place the rod instrument with this sponge in between each digit (toe) for sanitation purposes, dermabrasion, and/or the application of medicines & creams.

There are two (2) circular discs permanently attached to the applicator tip. These two (2) discs are the same in composition, size and diameter. The sole purpose of the two (2) discs is to hold the detachable/replaceable sponge applicators in place on the applicator tip, which is soldered to the instrument rod, during use and storage. The length between the two (2) discs is proportionate to the length of the sponge applicators, therefore, holding the sponge applicator in place horizontally on the applicator tip. The diameter of the discs is wider than the diameter of each individually sized sponge, therefore, maintaining the correct placement of the sponge applicator on the applicator tip during use and storage.

There will be two (2) hinge type attachments soldered to the instrument rod, one hinge at each end of the instrument rod. One hinge will permanently connect the frame and mirror to the instrument rod. The second hinge will permanently connect the applicator tip, discs and sponge applicators to the instrument rod.

1. What I claim as my invention is an instrument comprised of a long titanium and/or composite material rod. This long rod will have a magnified mirror attached at one end and an applicator tip, which will hold the applicator sponges, at the other end. This will provide the desperately needed ability for diabetic, arthritic, disabled, elderly and obese persons to self care for their medical and podiatric related conditions. My instrument will enable these same persons the ability to self examine and self medicate the tissues surrounding their feet, ankles and digits (toes) from a standing, sitting and/or lying position. My instrument will support the amount of pressure required for the user(s) to manually maneuver and manipulate the instrument rod for examination, dermabrasion, removal of tissue debris, sterilization, sanitation, as well as the application of prescribed medications and creams, sterilization liquids, over the counter medications and creams.
To check for ulcers and sores, diabetic persons must examine their feet and between their digits (toes) on a daily basis. To reach and bend down for an examination of the tops, sides and bottoms of the feet and digits (toes) can be an extremely difficult, if not impossible, process for these same persons, as well as arthritic, disabled, elderly and obese persons. The same reaching and bending problem exists when these same persons must remove tissue debris, sterilize, sanitize and/or medicate their feet, ankles and digits (toes).
From a distance, my inventions instrument rod will provide these same individuals, and all others, the ability to self examine the tissues surrounding their feet, ankles and the digits (toes) without any extensive reaching and/or bending. This self examination process can be performed solely through the use of my invention's extended instrument rod and the magnified glass mirror which is permanently attached, via soldered hinge, to one end of the instrument rod. The extended length of the instrument rod allows for the accurate positioning of the attached mirrored glass. The magnification of the mirrored glass allows the user(s) to visually inspect the medical, sterile and sanitary conditions of their feet, ankles and/or digits (toes).
From a distance, my inventions instrument rod will provide these same individuals, and all others, the ability for the sterilization, the medication, dermabrasion and the sanitation of their own feet, ankles and/or digits (toes). This process can be performed solely through the use of my invention's sponge applicators and the applicator tip which is permanently attached, via a soldered hinge, to the other end of the extended length of the instrument rod. I have invented the sponge applicators to be composed of a foam type sponge that will both absorb liquids from infections, for removal, and medications for application. In order to save money for the disabled, elderly and all others, I have invented these sponge applicators in a foam type sponge that is both washable and reusable.
For arthritic, elderly and all other persons, there is a soft rubber type grip placed in the center of the instrument rod, therefore, providing a non-slip, comfortable surface while maneuvering this device. This soft rubber type grip has been centered on the instrument rod in order to provide the same non-slip, comfortable surface regardless of whether the mirrored glass or the applicator tip and sponge procedures are been used.

2. What I claim as my invention are foam sponge applicators, in two (2) different sizes, which are both absorbent, washable and designed exclusively to fit the applicator tip as described in claim 3. This claim will be known referred to and known as: applicator sponge. My sponge applicators, both sizes, are composed of a foam type sponge that will both absorb liquids from infections, for removal, and medications for application.
These two (2) sponge applicators are the same size in length, however, they differ in their diameter. The first sponge maintains a larger diameter and is used for the removal of tissue debris, dermabrasion, sanitation, sterilization and the application of medicines and creams on ulcers and sores located on the foot and ankles surfaces. The second sponge maintains an extremely narrow diameter and is used for the removal of tissue debris, dermabrasion, sanitation, sterilization and the application of medicines and creams on ulcers and sores located between the digits (toes) and on the bottom of the digits (toes).
These sponge applicators were invented to be easily placed and secured on “The Medical Foot Helper For Diabetic, Arthritic, Disabled, Elderly and Obese Persons”. In order to save money for the disabled, elderly and all others, I have invented these sponge applicators in a foam type sponge that is both washable and reusable.

3. What I claim as my invention is a circular rod composed of titanium and/or composite material, approximately eight (8) inches in length, which was exclusively designed to hold in place and secure the applicator sponges, as stated in claim 2. This claim will be referred to and known as: applicator tip.

4. What I claim as my invention is a magnified glass mirror, secured within a hinged frame and attached to end of the long titanium and/or composite material rod, which I invented to exclusively fit my invention entitled “The Medical Foot Helper For Diabetic, Arthritic, Disabled, Elderly and Obese Persons”. This magnified glass mirror is approximately 6″×8″ in size, contained in and surrounded by a frame which has been permanently soldered to an extended length rod.
The magnified glass mirror and frame, which is attached to an extended length of rod, enables the user(s) to visually inspect their feet, ankles and digits (toes) without having to reach and/or bend over.
This magnified glass mirror and frame, which is attached to an extended length of rod, enables the user(s) to visually inspect their feet, ankles and digits (toes) while in a standing, sitting and/or lying position.
This magnified glass mirror and frame, which is attached to an extended length of rod, enables the user(s) to detect both large and small ulcers, sores, cuts, tissue debris, sanitation needs and sterilization needs.
